(19) United States
(12) Patent Application Publication
(10) Pub. No.: US 2005/0245463 A1
(43) Pub. Date: Nov. 3, 2005

(54) Novel antiretroviral sulfolipids extracted from spirulinae, method for obtaining same, compositions containing same and use thereof as inhibitors of the hiv virus reverse transcriptase

(76) Inventors: Quoc Kiet Pham, rue du Haut du Parc (FR); Hubert Durand-Chastel, rue Bobillot (FR)

Correspondence Address: DENNISON, SCHULTZ, DOUGHERTY & MACDONALD, 1727 KING STREET, SUITE 105, ALEXANDRIA, VA, 22314, US

(21) Appl. No.: 10/512,858

(22) PCT Filed: May 28, 2003
(86) PCT No.: PCT/FR03/01612

§ 371 Date: Nov. 15, 2004

(30) Foreign Application Priority Data
May 29, 2002 (FR) 02/06546

(51) Int. Cl. A61K 35/80; A61K 31/7024

(52) U.S. Cl. 514/24; 424/195.17; 536/4.1

(57) ABSTRACT

The invention relates to novel sulfolipids extracted from spirulins, to the process for obtaining them, to the compositions containing them and to their use as HIV-1 and HIV-2 inhibitors. The invention further relates to a spirulin biomass rich in sulfolipids which has an inhibitory activity towards HIV-1 and HIV-2 reverse transcriptase, and to the use of said sulfolipids, or of an extract of spirulin biomass rich in sulfolipids, for the preparation of a drug for the prophylactic or curative treatment of AIDS.

The invention relates to novel antiretroviral sulfolipids extracted from spirulins, to the process for obtaining them, to the compositions containing them, to their use as inhibitors of the human immunodeficiency viruses HIV-1 and HIV-2, and to the biomass containing them.

Glycolipids are very widespread in eukaryotic or prokaryotic organisms, in which they are associated with the membranes of thylacoids. In cyanobacteria in general, glycolipids are also associated with the cell walls of heterocytes (1). Cyanobacteria such as spirulins possess four types of membrane lipids: three glycolipids (two galactolipids and one sulfolipid) and a single type of phospholipid (phosphatidylglycerol).

Several studies have shown that these glycolipids can have anti-inflammatory, antitumor or antiviral activities (2). Gustafson et al. (3) have studied the antiviral activity towards HIV-1 of sulfolipids extracted from the microalgae Lyngbya lagerheimii and Phormidium tenue: The results show an HIV-1 inhibitory activity of pure prokaryotic sulfolipids in a tetrazolium test as well as in tests relating to the formation of syncitia and to protein P24 in human lymphocyte cell lines.

Recently, Loya et al. (4) have shown that prokaryotic sulfolipids in Scytonema sp., Oscillatoria trichoides, Oscillatoria raoi, Oscillatoria limnetica and Phormidium tenue are powerful inhibitors of HIV-1 reverse transcriptase, which is considered to be a key enzyme in the life cycle of HIV.

Reverse transcriptase is a multifunctional enzyme having two enzymatic activities, namely DNA polymerase and RNAse-H activities. These two activities are responsible for the conversion of viral genomic DNA to proviral double-stranded DNA. This DNA is then transported from the cytoplasm into the nucleus of the host cell, where it is subsequently integrated into the cellular DNA. Inhibition of each of the two catalytic functions of reverse transcriptase prevents viral production in the host cell, so this enzyme is one of the principal targets in the search for AIDS treatments.

In the search for AIDS treatments, attempts are also made to inhibit the other essential steps of infection by the virus, namely:
binding of the virus to the infected cell;
fusion of the membranes of the virus and the infected cell;
integration of the proviral DNA into the DNA of the host cell with the aid of integrase;
remodeling of the viral proteins to produce a new virus under the effect of the viral protease.

Drug combinations are being studied in order to control the development of the infection as far as possible.

There are two types of reverse transcriptase inhibitor:
1) nucleoside inhibitors of the AZT type: ddI, ddC, 3TC, d4T, abacarir;
2) non-nucleoside inhibitors: sulfolipids and other molecules (nevirapine, efavirenz, etc.).

The sulfolipids already described as reverse transcriptase inhibitors are prokaryotic sulfolipids extracted from the microalgae Lyngbya lagerheimii and Phormidium tenue, which inhibit the cytopathic effects of HIV-1 (3), these sulfolipid compositions described in WO 91/02521 and the C18/C16 and C16/C16 prokaryotic sulfolipids obtained from Oscillatoria raoi, O. trichoides, O. limnetica, Scytonema sp. and Phormidium tenue by Loya S. et al. (21).

Sulfolipids are also constituents of spirulins, which are blue-green microalgae of particular nutritional value to malnourished children. Rich in compounds of nutritional and biomedical value, such as essential amino acids, vitamins (A, B12, E) or essential polyunsaturated fatty acids, they develop mainly in the sodium-containing waters of a number of tropical lakes in the arid belt.

These microalgae belong to Phyllum cyanophyta, class: Cyanophyceae, order: Nostocales, family: Oscillatoriaceae, genus: Spirulina or Arthrospira.

Different species thereof exist, particularly the species Spirulina platensis and Spirulina maxima (Bourrelly P. 1970. Les algues bleues ou cyanophycees (Blue algae or Cyanophyceae), in “Les Algues d'eau douce” (“Freshwater algae”), volume III, published by N. Boubee).

Patent FR 2 768 744 in the names of the Co-applicants describes a process for the mixotrophic culture of spirulins for the production of a biomass rich in omega 6 polyunsaturated fatty acids and/or sulfolipids. This process comprises at least one step for the culture of spirulins in the presence of ammonium linoleate.

Pursuing their research studies, the Co-applicants have now found that it is possible to isolate a group of sulfolipids of the prokaryotic type and eukaryotic type from an extract of spirulin cultivated in the presence of ammonium oleate or ammonium palmitate, said sulfolipids having an improved activity as HIV-1 and HIV-2 reverse transcriptase inhibitors.

In cyanobacteria in general and spirulin in particular, the typical distribution of fatty acids over the glycerol backbone of the lipids (galactolipids, phospholipids and sulfolipids) corresponds to C18 and C16 fatty acids esterified on carbons 1 and 2, respectively. This distribution characterizes the C18/C16 and C16/C16 molecular species referred to as “prokaryotic”, which are more or less unsaturated.

In the remainder of the description, “sulfolipids of the prokaryotic type” (or “prokaryotic sulfolipids”) are understood as meaning the sulfolipids of formula (I): 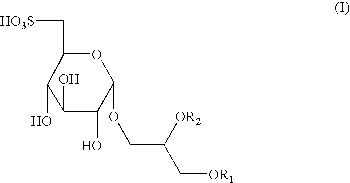
in which R₁ is a C₁₈ unsaturated fatty acid radical or a C₁₆ saturated or unsaturated fatty acid radical and R₂ is a C₁₆ saturated or unsaturated fatty acid radical, and “sulfolipids of the eukaryotic type” (or “eukaryotic sulfolipids”) are understood as meaning the sulfolipids of the above formula in which R₁ and R₂ are identical or different C₁₈ unsaturated fatty acids, i.e. C18/C18 sulfolipids.

“Saturated fatty acid radical” is understood as meaning a hydrocarbon chain not comprising a double bond.

“Unsaturated fatty acid radical” is understood as meaning a hydrocarbon chain containing one or more double bonds, preferably 1, 2 or 3 double bonds.

Surprisingly, it has now been found that supplementing the spirulin culture medium with ammonium oleate or ammonium palmitate modifies the composition of the total sulfolipids and increases the sulfolipids of the eukaryotic type and prokaryotic type, as defined above, thereby increasing the inhibitory activity towards HIV-1 and HIV-2 reverse transcriptase of the sulfolipids extracted from spirulins cultivated in supplemented medium.

According to a first feature, the invention therefore relates to a novel process for the culture of spirulins in which the culture medium is supplemented with exogenous fatty acids in the form of ammonium oleate or palmitate so as selectively to increase certain sulfolipid molecular species.

This biomass is used to extract the lipids. The lipid classes are then separated in order to harvest the total sulfolipids. These are then separated into the different sulfolipid molecular species.

The invention further relates to said sulfolipids, to the compositions containing them, to their use as HIV-1 and HIV-2 reverse transcriptase inhibitors, and to their use for the preparation of a drug for the treatment of AIDS.

The culture process according to the invention applies to all the existing strains of spirulins, especially those described in the publications cited earlier. The strain used can be selected e.g. from the following strains:
Spirulina platensis PCC 8005 (Institut Pasteur, Paris);
Spirulina maxima and Spirulina texcoco (Texcoco, Mexico);
Spirulina crater (Laboratoire La Roquette, France);
Spirulina 8818 (ENS, Paris).

Spirulins grow fairly well in culture media supplemented with ammonium linoleate. They absorb exogenous linoleic acid in the form of ammonium linoleate to synthesize γ-linolenic acid in their lipids such as monogalactosyldiacylglycerol (MGDG), digalactosyldiacylglycerol (DGDG), sulfoquinovosyldiacylglycerol (SQDG) and phosphatidylglycerol (PG).

It has now been found that specific culture conditions, using ammonium oleate or palmitate as supplement under optimized temperature and illumination conditions, make it possible to obtain a spirulin biomass particularly rich in sulfolipids that inhibit HIV-1 and HIV-2 reverse transcriptase.

The spirulin biomass can be produced in a tank or a sterile photobioreactor. Appropriate tanks and photobioreactors for this type of culture are well known to those skilled in the art.

According to a preferred feature, the invention therefore relates to a process for the mixotrophic culture of spirulins for the production of a biomass rich in sulfolipids that inhibit HIV-1 and HIV-2 reverse transcriptase, said process comprising at least one step for the culture of spirulins in the presence of ammonium oleate or ammonium palmitate.

Preferably, the concentration of ammonium oleate or ammonium palmitate added to the medium is between 35 and 75 μmol/l.

Advantageously, the temperature during the culture step in the presence of ammonium oleate or ammonium palmitate is 20° C. to 30° C. Preferably, the illumination during said step is between 100 and 125 μE/m²/s with a 24 h alternating illumination cycle of 8 to 12 h of white light and 16 to 12 h of darkness, preferably 12 h of white light and 12 h of darkness.

Particularly advantageous culture conditions for the production of a spirulin biomass rich in sulfolipids that inhibit HIV-1 and HIV-2 reverse transcriptase consisting in:
bubbling 25 to 60 l of air enriched with 1% (by volume) of CO₂/l of medium/h;
maintaining the pH of the culture medium at between 8.5 and 10.5, preferably from 9 to 10, during growth in order to avoid contamination by other microorganisms unable to develop in a very basic medium;
maintaining the minimum concentrations of bicarbonate, phosphate and nitrate ions that are appropriate to the needs of the spirulin strain during growth.

According to one advantageous feature, said process comprises the steps consisting in:
supplementing the medium with ammonium oleate or ammonium palmitate under an illumination of 75 to 100 μE/m²/s with a 24 h alternating illumination cycle of about 8 to 12 h of white light and about 16 to 12 h of darkness, preferably about 12 h of white light and about 12 h of darkness, the temperature being maintained at about 30° C., for 48 h;
then maintaining an illumination of 100 to 125 μE/m 2/s at 24° C. for 48 to 72 h with a 24 h alternating illumination cycle of about 8 to 12 h of white light and about 16 to 12 h of darkness, preferably about 12 h of white light and about 12 h of darkness;
then maintaining an illumination of 100 to 125 μE/m²/s at 22° C. for 72 to 96 h, it being possible for said illumination to be maintained up to 168 h (i.e. 7 days) at 20° C., with a 24 h alternating illumination cycle of about 8 to 12 h of white light and about 16 to 12 h of darkness, preferably about 12 h of white light and about 12 h of darkness, optionally with the bubbling of air enriched with 1% of CO₂ at a rate of 25 l/l of culture/h for 24 to 48 h after supplementation with ammonium oleate or ammonium palmitate, and then at a rate of 40-60 l/l of culture/h for 48 to 96 h.

According to a final feature, the invention further relates to a spirulin biomass rich in sulfolipids that contains at least 40% by weight of sulfolipids, based on the total lipids, said sulfolipids having an inhibitory activity towards HIV-1 and HIV-2 reverse transcriptase.

The sulfolipids contained in the biomass are prokaryotic sulfolipids or eukaryotic sulfolipids.

Preferably, said sulfolipids have formula (I) below: 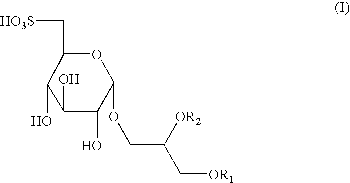
in which:
R₁ is an oleoyl radical and R₂ is a palmitoyl radical, or
R₁ is a linoleoyl radical and R₂ is a palmitoyl radical, or
R₁ is a palmitoyl radical and R₂ is a palmitoyl radical, or
R₁ is a γ-linolenoyl radical and R₂ is a palmitoyl radical, or
R₁ is a γ-linolenoyl radical and R₂ is a palmitoleoyl radical, or
R₁ is a palmitoleoyl radical and R₂ is a palmitoyl radical.

According to the nomenclature defined above, these sulfolipids are sulfolipids of formula (I) in which R₁ and R₂ are defined as follows:

| R₁ | R₂ |
|---|---|
| C18:1 | C16:0 |
| C18:2 | C16:0 |
| C16:0 | C16:0 |
| γC18:3 | C16:0 |
| γC18:3 | C16:0 |
| C16:1 | C16:0 |

Other advantageous sulfolipids are the eukaryotic sulfolipids of formula (I) in which R₁ is a C₁₈ unsaturated fatty acid radical and R₂ is a C₁₈ unsaturated fatty acid radical, said radicals being identical or different.

Among these, advantageous sulfolipids are those of formula (I) in which:
R₁ is an oleoyl radical and R₂ is a linoleoyl radical, or
R₁ is a linoleoyl radical and R₂ is an oleoyl radical, or
R₁ is a linoleoyl radical and R₂ is a linoleoyl radical, or
R₁ is an oleoyl radical and R₂ is an oleoyl radical.

These eukaryotic sulfolipids have formula (I) in which R₁ and R₂ are defined as follows:

| R₁ | R₂ |
|---|---|
| C18:1 | C18:2 |
| C18:2 | C18:1 |
| C18:2 | C18:2 |
| C18:1 | C18:1 |

The invention further relates to mixtures containing eukaryotic and/or prokaryotic sulfolipids defined above, which are also called “total sulfolipids”.

Advantageously, said sulfolipids are isolated from the spirulin biomass rich in sulfolipids, described above, by steps for the extraction, separation and purification of the different sulfolipid molecular species, and represent a further feature of the invention.

The lipid compounds can be extracted e.g. with solvents such as methanol and chloroform. Separation can be performed by techniques known to those skilled in the art, such as thin layer chromatography or high performance liquid chromatography. Different sulfolipid molecular species are preferably separated by high performance liquid chromatography.

According to another one of its features, the invention relates to the sulfolipids of formula (I) as defined above.

The invention further relates to the use of said sulfolipids, or of an extract of spirulin biomass rich in prokaryotic and eukaryotic sulfolipids, as defined above, as HIV-1 or HIV-2 reverse transcriptase inhibitors.

The invention further relates to pharmaceutical compositions containing said sulfolipids in association with a pharmaceutically acceptable vehicle, and to the use of said sulfolipids, or of an extract of spirulin biomass rich in sulfolipids, for the preparation of a drug for the prophylactic or curative treatment of AIDS.

The invention is illustrated by the following Examples without implying a limitation.

### EXAMPLE 1

### Culture of Spirulin in the Presence of Exogenous Fatty Acids (Ammonium Oleate or Palmitate) to Give the Biomass Rich in Sulfolipids

The strain used is Spirulina platensis PC 8005 (Institut Pasteur, Paris, France).

Process for the Culture of Spirulin Rich in Sulfolipids in a Photobioreactor in the Presence of Ammonium Oleate or Palmitate

a) Multiplication of Spirulina platensis

Strains are multiplied as described in Example 3.1 of patent FR 2 767 744.

b) 5 l of culture are prepared in the 1st photobioreactor (capacity 7 l) by the steps mentioned in Example 3.2) a) of FR 2 768 744.

c) 4 l of culture are withdrawn from the 1st photobioreactor, said photo-bioreactor being made up with 4 l of fresh Zarrouk sterile medium (having the composition given in FR 2 768 744) in order to continue preparing another culture. 4 l of this culture are then transferred under sterile conditions into the second 7 l photobioreactor as inoculum, the medium being supplemented with ammonium oleate . . . at a concentration of 35-75 μmol of ammonium oleate or palmitate/l for 5-7 days, under the following culture conditions:

c.1) The first step comprises 2 successive phases:
Phase 1: The culture is maintained at 30° C. under an illumination of 75 to 100 μE/m²/s for 48 h. At the same time the 24 h illumination cycle has to be set to about 8-12 h of white light/16-12 h of darkness. In addition, the bubbling of air enriched with 1% of CO₂ has to be reduced and maintained at a rate of 25-35 l/l of culture/h for 24 to 48 h. The stirring speed is maintained at about 100-150 rpm.
Phase 2: The culture is placed at 24° C. under a stronger illumination of 100 to 125 gE/m²/s for 48 h. The 24 h illumination cycle is about 8-12 h of white light/16-12 h of darkness. The bubbling of air enriched with 1% of CO₂ is increased and maintained at 40-50 l/l of culture/h for 48-72 h. The stirring speed is maintained at about 100-150 rpm.

c.2) In the second step the culture temperature is lowered to 20-22° C. under an illumination of 100-125 μE/mm²/s. The 24 h illumination cycle is about 12 h of white light/12 h of darkness for 72-96 h before the biomass is harvested. The pH is about 9-10.5 in order to optimize the synthesis of sulfolipid in the spirulin cells. The culture is aerated with a mixture of air enriched with 1% of CO₂ at a rate of 50-60 l/l of culture/h. The stirring speed is maintained at about 100-150 rpm. The duration of the second step can vary according to the strain cultivated and the type of photobioreactor used.

d) The biomass rich in sulfolipids is harvested by the following process:

The spirulin culture is maintained at 20-24° C. for 24-48 h in the decanters under an illumination of 30-50 μE/m²/s in order to remove the supernatant. The biomass precipitates to the bottom of the decanters and is harvested by filtration or centrifugation at 5000 rpm for 15 min and then rinsed with an NaCl solution containing 10 g/l at 24° C. The biomass is subsequently harvested by further centrifugation at 5000 rpm for 15 min and then rinsed 3 times with distilled or demineralized water at 20-24° C. prior to lyophilization or atomization.

e) Results

The proportion of total lipids in the spirulins cultivated by the process of the invention is about 6.7-7.2% of the dry weight. The culture yield reaches 1.6 to 2.1 g of dry weight/l. Increasing the initial cellular concentration makes it possible to reduce the culture time while increasing the production yield (from 2.2 to 2.6 g/l).

e.1) Lipid composition of the spirulins cultivated in the presence of ammonium oleate, linoleate or palmitate (% by weight)

The proportion of sulfolipids obtained is about 38-41.5% of the total lipids, as indicated in Table 1 below.

TABLE 1

| Culture | MGDG¹ | DGDG² | PG³ | SQDG⁴ |
|---|---|---|---|---|
| Control⁵ | 31-33 | 15.7-17.3 | 24.8-26.2 | 24.8-26.6 |
| +oleate | 34-35 | 10-10.8 | 13-13.5 | 40-41.5 |
| +palmitate | 40.0-43.5 | 8.5-9.3 | 10-11 | 38.0-39.7 |

¹monogalactosyldiacylglycerol
²digalactosyldiacylglycerol
³phosphatidylglycerol
⁴sulfoquinovosyldiacylglycerol
⁵without additive

The results show that the proportion of sulfolipids in the form of sulfoquinovosyldiacylglycerol is significantly increased when the culture medium is supplemented with ammonium oleate or palmitate.

e.2) Total fatty acid composition and fatty acid composition of the sulfolipids in the case of S. platensis PC 8005 cultivated either without additive or in the presence of ammonium oleate or palmitate (Tables 2 and 3)

The sulfolipids have the formula below:

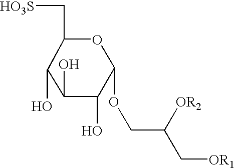

in which R₁ and R₂ are defined as follows:

| R₁ | R₂ |
|---|---|
| γC18:3 (γ-linolenoyl) | C16:0 (palmitoyl) |
| C18:2 (linoleoyl) | C16:0 (palmitoyl) |
| C18:1 (oleoyl) | C16:0 (palmitoyl) |
| C16:1 (palmitoleoyl) | C16:0 (palmitoyl) |

The formulae corresponding to these definitions of R₁ and R₂ are as follows:

| | | |
|---|---|---|
| palmitoyl | 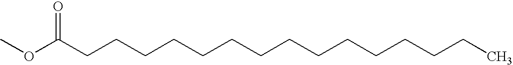 | C16:0 |
| palmitoleoyl | 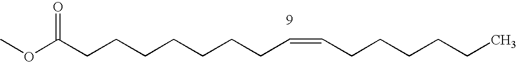 | C16:1 |
| oleoyl | 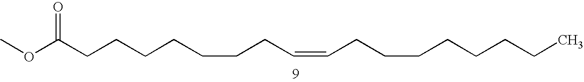 | C18:1 |
| linoleoyl | 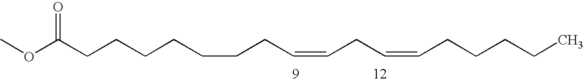 | C18:2 |
| γ-linolenoyl | 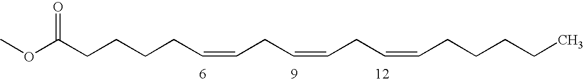 | γC18:3 |

TABLE 2
Total fatty acid composition (% by weight)*

| | | | | | | | Ratio |
|---|---|---|---|---|---|---|---|
| Culture | 16:0 | 16:1 | 18:0 | 18:1 | 18:2 | γ18:3 | C16/C18 |
| control | 50.2 | 1.2 | 1.3 | 10.5 | 16.0 | 20.8 | 51.4/48.6 |
| +oleate | 34.7 | 1.2 | 1.0 | 20.1 | 18.0 | 25.0 | 35.9/64.1 |
| +palmitate | 55.4 | 1.9 | 1.6 | 3.7 | 12.8 | 24.6 | 57.3/42.7 |

*standard error ≦ 0.3

The results show that supplementing the medium with ammonium oleate significantly increases the C18 total fatty acids and decreases the C16 total fatty acids.

Supplementing with ammonium palmitate causes a slight increase (6%) in the C16 total fatty acids and a decrease in the C18 total fatty acids.

TABLE 3
Fatty acid composition of the sulfolipids (% by weight)*

| | | | | | | | Ratio |
|---|---|---|---|---|---|---|---|
| Culture | 16:0 | 16:1 | 18:0 | 18:1 | 18:2 | γ18:3 | C16/C18 |
| control | 55.8 | 1.0 | 1.0 | 11.1 | 27.0 | 4.1 | 56.8/43.2 |
| +oleate | 45.2 | 1.5 | 0.5 | 18.6 | 29.0 | 5.2 | 46.7/53.3 |
| +palmitate | 58.9 | 1.9 | 1.0 | 9.0 | 25.5 | 4.7 | 60.8/39.2 |

*standard error ≦ 0.4

The results show that supplementing the medium with ammonium oleate significantly increases the C18 fatty acids and decreases the C16 fatty acids in the sulfolipids.

Supplementing with ammonium palmitate has the opposite effect.

This shows that, in the presence of oleate, the spirulin utilizes this exogenous fatty acid to synthesize eukaryotic (C18/C18) sulfolipids preferentially, whereas, in the presence of ammonium palmitate, the synthesis of prokaryotic (C16/C16) sulfolipids is increased.

### EXAMPLE 2

### Extraction, Separation and Purification of the Sulfolipid Molecular Species

2.1. Extraction and Separation of the Lipid Classes by TLC (Thin Layer Chromatogranhy) or HPLC (High Performance Liquid Chromatography)

2.1.1. Extraction

The lipids are extracted by the method of Bligh and Dyer (1959) with methanol and chloroform (5). The chloroform phase is withdrawn, dried under nitrogen and then taken up in a volume of chloroform or benzene/ethanol (4:1, v/v).

2.1.2. Separation

a) By TLC:

The total lipid extract is deposited under nitrogen on a 0.25 mm thick silica gel plate (Silicagel G60, Merck). One-dimensional migration is carried out in a hermetically sealed tank containing a mixture of chloroform/acetone/methanol/acetic acid/bi-distilled water (50:20:10:10:5, v/v) (6). The spots are developed by spraying with distilled water, the lipid controls being developed by spraying with a primulin solution (10 mg/10 ml of 80% acetone in water) and observing under UV. The spots are collected and recovered in tubes containing a mixture of chloroform/methanol/water (2; 1:0.5, v/v). The samples are placed at −20° C. for 24 h and the lipid fraction (chloroform phase) in each tube is then harvested and evaporated to dryness under vacuum on a rotary evaporator or under nitrogen. Finally, the lipid classes are redissolved in a known volume of chloroform for analysis of the molecular species.
b) By HPLC:

The lipid extract, filtered through a Millipore® membrane (diameter 0.5 μm), is evaporated to dryness under nitrogen and then dissolved in 100 μl of chloroform. The lipid categories are separated on a Waters HPLC set-up (Milford, Ma, USA) with a 300×7.8 mm Parasil 10 gum silica column according to Demandre et al. (7, 8), the lipid extract being eluted first for 2 min with a solvent A comprising a mixture of isopropanol and hexane (4:3, v/v). Then the lipids are eluted for 20 min with a mixture of two solvents according to a linear gradient starting at 100% of solvent A and ending at 100% of solvent B, the latter comprising isopropanol/hexane/H₂O (8:6:1.5, v/v/v). The column is finally eluted for 20 min with solvent B at a rate of 2 ml/min. The lipids detected at 205 nm are collected. They are identified by thin layer chromatography according to the Lepage method (6) using controls (MGDG, DGDG, SQDG and PG) and specific reagents, including x-naphthol and sulfueric acid for the galactolipids and Zinzadze reagents for the phospholipids (9).

The lipid classes can be redissolved in ethanol for HIV experiments.

2.1.3. Assay of the fatty acids and lipids

The lipid spots on the silica gel plate are scratched off for methylation of their fatty acids. Methylation of the fatty acids of the total lipid extract or the fatty acids of the lipid classes separated by TLC is carried out in the presence of a C17:0 internal standard (heptadecanoic acid). 3 ml of methanol/sulfuric acid (97.5:2.5, v/v) are then added to the sample (10). After 40 min at 75° C. in a sealed tube, the sample is immediately cooled and the methyl esters are extracted with 2 ml of hexane and 1 ml of bi-distilled water.

The methyl esters are analyzed on a Hewlett Packard gas chromatograph. The amount of each fatty acid is calculated by comparison with the C17:0 internal standard.

2.2. Separation of the Different Sulfolipid Molecular Species by HPLC

2.2.1. Separation of the Sulfolipid Molecular Species by HPLC

The sulfolipid obtained by TLC or HPLC is separated into molecular species on a reversed phase column. The stationary phase used is either the ODS 5 μm phase (in a 4.6×250 mm Altex or Spherisorb column) eluted with a solvent of the following composition: methanol/acetonitrile/H₂O (90.5:2.5:4, v/v/v) containing 20 mM choline chloride, or the Bondapak C18 10 μm phase (in a 3.9×300 mm Waters column) eluted with a solvent consisting of a mixture of methanol/acetonitrile/H₂O (90.5:2.5:7, v/v/v) containing 20 mM choline chloride. The solvent flow rate is 1.5 ml/min. The sulfolipid molecular species are separated and simultaneously detected together at 205 nm. Analysis of the fatty acids of the sulfolipid molecular species by GC enables said species to be identified and quantified (7).

2.2.2. Identification of the Prokarvotic and Eukaryotic Sulfolipid Molecular Species

The sulfolipid molecular species are identified by analysis of the position of the fatty acids on the glycerol of the sulfolipid molecules.

The sulfolipids separated by TLC or HPLC are scratched off and wetted with bi-distilled water. They are then extracted three times with a mixture of methanol/chloroform (1:2, v/v) and once with pure methanol. The sulfolipid extract is dried under nitrogen.

The method used is that of Fischer et al. (1973) (14): 20 ml of Triton X-100 (100 mg of Triton X-100 in 2 ml of chloroform) and 100 μl of chloroform are added to the dried sample. 1 ml of 0.04 M Tris-HCl buffer (pH=7.5) and 20 μl of lipase A1 from Rhizopus arrhizus (50,000 U/ml, Boehringer) are then introduced into the tube containing the redried sample. After vigorous stirring with a Vortex for 1 to 2 min, the tubes are incubated in a gently stirred water bath at 30° C. for 30-60 min for analysis of the sulfolipids and phospholipids (20-30 min for the galactolipids). Lipase A1 hydrolyzes exclusively the ester group located in the 1-position of the glycerol. The reaction is stopped in ice by the addition of 15 μl of 1.8 N acetic acid or isopropanol. The solvent is evaporated off under nitrogen and 3 ml of chloroform/methanol (1:1, v/v) are added to the hydrolyzed sample. After vigorous stirring, the hydrolyzed sample is centrifuged at 400 g for 10 min and the supernatant is taken up for analysis by TLC. The supernatant contains the products hydrolyzed by lipase A1 (free fatty acids and 2-acyl-lyso SQDG), which are separated on a thin layer of silica (Silicagel G60, Merck) with Lepage solvent (6) comprising a mixture of chloroform/acetone/methanol/acetic acid/H₂O (50:20:10:10:5, v/v/v/v/v). The solvent used for the sulfolipid derivatives is a mixture of chloroform/methanol/acetic acid/H₂O (65:35:4:4 by volume). After migration, the free fatty acids originating from the 1-position of the glycerol and the 2-acyl-lyso SQDG, developed with a primulin solution, are scratched off and analyzed by gas chromatography after methylation in the presence of 1% sodium methylate (0.2 ml) and methanol/1.1 N hydrochloric acid (0.2 ml) (or methanol/sulfuric acid (97.5:2.5)).

2.3. Purification of the Prokarvotic (C18/C16) and Eukaryotic (C18/C18) Sulfolipid Molecular Species

A Seppaks silica cartridge can be used to separate the neutral lipids, the galactolipids, the phospholipids and the sulfolipids.

The neutral lipids are removed with chloroform, while the galactolipids and phospholipids are subsequently separated off with a mixture of methylene chloride/methanol (93:7, v/v) and methanol, respectively. After this solvent system has been used, the sulfolipids are in dilute form in the phospholipid fraction (methanol). The sulfolipids are then separated from the phospholipid fraction (methanol fraction) by normal phase HPLC on a MAXISIL 5 μm SI column (150×10 mm) (Phenomenex, Torrance, Calif.).

The mobile phase is a mixture of heptane/isopropanol/0.001 M KCl (40:52:8, v/v/v) at a flow rate of 1.5 ml/min. The sulfolipid peaks are found by detection at 208 nm (after 25 min of dilution). Finally, the column is washed with 100% isopropanol and re-equilibrated with 100% heptane after each operation (15).

The results are reported in Table 4 below.

TABLE 4
Composition of the sulfolipid molecular species
(% by weight of total sulfolipids)

| Molecular species | | | | | | | |
|---|---|---|---|---|---|---|---|
| prokaryotic | eukaryotic | Culture | | | | | |
| C18/C16 | C18/C18 | control | | +oletate | | +palmitate | |
| γC18:3/C16:1 | | 1.0 | | 1.0 | | 1.0 | |
| | γC18:3/C18:2 | | nd* | | nd | | nd |
| | C18:2/C18:2 | | nd | | nd | | nd |
| γC18:3/C16:0 | | 0.8 | | 1.5 | | 1.0 | |
| | C18:2/C18:1 | | nd | | 5.6 | | nd |
| | C18:1/C18:2 | | nd | | 3.4 | | nd |
| | C18:1/C18:1 | | nd | | 0.5 | | nd |
| C18:2/C16:0 | | 76.9 | | 76.4 | | 73.2 | |
| C18:1/C16:0 | | 5.5 | | 10.1 | | 4.1 | |
| C16:0/C16:0 | | 15.8 | | 2.0 | | 20.0 | |
| C16:1/C16:0 | | nd | | nd | | 0.7 | |

| | | | |
|---|---|---|---|
| Ratio of total C18/C16/ | 100/0 | 91/9.0 | 100/0 |
| total C18/C18 | | | |

*not determined

The results show that the composition of the sulfolipid molecular species differs according to the supplementation of the medium. In fact, in the ratio of total C18/C16/total C18/C18, the proportion of C18/C16 (prokaryotic) sulfolipids decreases and the proportion of C18/C18 (eukaryotic) sulfolipids increases significantly when the culture medium is supplemented with ammonium oleate.

### EXAMPLE 3

### Inhibition of HIV-1 and HIV-2 Reverse Transcriptase by the Sulfolipid Molecular Species of Spirulin

3.1. Test Method

3.1.1. Enzymes

The reverse transcriptases used in this study are the recombinant enzymes expressed in E. coli and purified from the bacterial extracts (16). The HIV-1 reverse transcriptase expression plasmid originates from the proviral isolate BH-10 (17), while the HIV-2 reverse transcriptase expression plasmid originates from the isolate pRod (18). The HIV-1 and HIV-2 reverse transcriptases are the hetero-dimers p.66/p.5 l and p.68/p.55, respectively.

3.1.2. Enzymatic Tests

The abbreviations used are as follows:
dTTP: deoxythymidine triphosphates
dATP: deoxyadenosine triphosphates
dGTP: deoxyguanosine triphosphates
dCTP: deoxycytosine triphosphates
dNTPs: deoxynucleoside triphosphates

The enzymatic tests are performed by the method of Loya et al. (19). The DNA polymerase activity is measured by monitoring the incorporation of poly(rA)ₙ.oligo (dT)₁₂₋₁₈ in [³H] dTTP into the product insoluble in trichloroacetic acid (TCA), in the presence of different concentrations of sulfolipids. The RNase-H activity is measured by measuring the delivery of the TCA-soluble product from the synthetic substrate [³H]poly(rA)ₙ.poly (dT)ₙ. This substrate is prepared by the procedure of Hizi et al. (1991) (20). In all the inhibition experiments, the enzymes are preincubated for 5 min at 30° C. in the absence or presence of inhibitors at different concentrations. The enzymatic reactions are started by the addition of appropriate substrate at 37° C. for 30 min.

The residual enzymatic activity is calculated relative to the initial rates of the (linear) reaction observed in the absence of sulfolipids.

The concentration of inhibitors that leads to 50% inhibition of the enzymatic activities (IC₅₀) is calculated from the curves of inhibition as a function of inhibitor (sulfolipid) concentration.

The enzymatic activities are defined as follows:

(a) One unit of DNA polymerase activity is the amount of enzyme which catalyzes the incorporation of one pmol of dNTP into the DNA product at 37° C. over 30 min under standard test conditions.

(b) One unit of RNase-H activity is the amount of enzyme which catalyzes the hydrolysis of one pmol of AMP at 37° C. over 30 min under standard test conditions.

3.2. Results

The results are reported in Tables 5, 6 and 7 below.

TABLE 5
Effect of the total sulfolipids on HIV-1 reverse transcriptase

| | Concentrations of | Residual enzymatic |
|---|---|---|
| Total sulfolipids | sulfolipids (μg/ml) | activity (%) |
| control | 332-374.7 (+4.7) | 10 + 2 |
| +oleate | 215-267.7 (+5.0) | 10 + 2 |
| +palmitate | 347.5-393.0 (+4.7) | 10 + 2 |

The results show that the inhibitory activity towards HIV-1 reverse transcriptase of the total sulfolipids from spirulin cultivated in the presence of oleate is significantly greater than that of the control.

In fact, HIV-1 reverse transcriptase is 90% inhibited at lower doses, namely 215 and 291.5 μg/ml, for the total sulfolipids extracted from spirulin cultivated in the presence of oleate, whereas the dose is 332 μg/ml for spirulin cultivated in non-supplemented medium.

This phenomenon can be explained by the modification of the composition of the total sulfolipids due to supplementation of the medium, namely the increase in prokaryotic (C18/C16) sulfolipids and the appearance of eukaryotic (C18/C18) sulfolipids.

TABLE 6
Inhibition of the DNA polymerase and the RNase-H of HIV-1
reverse transcriptase by the sulfolipid molecular species

| | | | RNase-H (b) |
|---|---|---|---|
| | DNA polymerase (a) | | % of initial |
| Molecular | IC₅₀ | IC₉₀ | enzymatic activity |
| species | μg/ml | μg/ml | (c) |

Prokaryotic:

| | | | |
|---|---|---|---|
| C18:1/C16:0 | 23.9 + 3.8 | 73.6 + 2.8 | 64 + 6 |
| C18:2/C16:0 | 75.4 + 2.9 | 281.4 + 4.8 | 100 |
| γC18:3/C16:0 | 178.0 + 12.4 | 533.1 + 13.0 | 100 |
| C16:0/C16:0 | 106.1 + 12.0 | 451.8 + 12.5 | 47 + 4 |
| C16:1/C16:0 | 87.4 + 3.3 | 302.0 + 3.2 | 42 + 3 |

Eukaryotic:

| | | | |
|---|---|---|---|
| C18:2/C18:2 | 279.0 + 24.6 | 840.1 + 22.7 | 100 |
| C18:2/C18:1 | 250.9 + 17.7 | 752.8 + 19.7 | 100 |
| C18:1/C18:2 | 209.4 + 19.7 | 628.3 + 24.6 | 100 |
| C18:1/C18:1 | 20.1 + 1.8 | 68.3 + 2.0 | 60 + 2 |

(a) IC₅₀ and IC₉₀: concentrations of the inhibitors (sulfolipids) that inhibit 50% and 90% of the initial enzymatic activity, respectively. The inhibitory concentrations are expressed in μg/ml.
(b) The RNase-H activity is measured in the presence of the inhibitors with a concentration of 100 μM of each sulfolipid molecular species.
(c) The residual enzymatic activity is calculated as the percentage of the control in the absence of the inhibitors.

The results show that the inhibitory activity towards the DNA polymerase of HIV-1 reverse transcriptase of the prokaryotic molecular species is greater than that of the eukaryotic species except C18:1/C18:1, the highest activities being those of the C18:1/C18:1, C18:1/C16:0 and C18:2/C16:0 species.

As regards inhibition of the RNase-H of HIV-1 reverse transcriptase, the highest activities are those of the C16:1/C16:0, C16:0/C16:0, C18:1/C18:1 and C18:1/C16:0 prokaryotic species.

TABLE 7
Effect of the sulfolipid molecular species on the DNA
polymerase activity of HIV-2 reverse transcriptase

| | Concentrations of | Residual enzymatic |
|---|---|---|
| Molecular species | sulfolipids (μg/ml) | activity (%) |
| C18:1/C16:0 | 4.8 + 0.9 | 100 + 1 |
| | 9.6 + 0.9 | 93 + 2 |
| | 19.1 + 0.9 | 74 + 2 |
| | 33.5 + 2.0 | 50 + 1 |
| | 38.3 + 1.8 | 42 + 2 |
| | 76.5 + 2.0 | 24 + 2 |
| C18:2/C16:0 | 9.5 + 0.9 | 100 + 1 |
| | 23.8 + 0.9 | 96 + 2 |
| | 47.7 + 1.8 | 70 + 2 |
| | 95.4 + 2.0 | 56 + 2 |
| | 105.0 + 5.0 | 50 + 1 |
| | 190.8 + 4.8 | 25 + 2 |
| C16:1/C16:0 | 10.2 + 0.5 | 100 + 1 |
| | 25.0 + 0.6 | 96 + 1 |
| | 48.9 + 0.9 | 69 + 1 |
| | 97.5 + 1.5 | 58 + 2 |
| | 108.0 + 3.0 | 50 + 1 |
| | 198.5 + 3.4 | 24 + 2 |
| C18:1/C18:1 | 4.0 + 0.5 | 100 + 1 |
| | 8.0 + 0.6 | 92 + 1 |
| | 16.0 + 0.7 | 70 + 2 |
| | 26.1 + 1.8 | 50 + 1 |
| | 36.0 + 1.0 | 35 + 1 |
| | 71.9 + 1.0 | 22 + 2 |

The results show that the C18:1/C16:0 and C18:2/C16:0 sulfolipid molecular species also have an inhibitory activity towards the DNA polymerase activity of HIV-2 reverse transcriptase, the activity of the C18:1/C18:1 sulfolipids being the highest.

### REFERENCES

(1) GAMBACORTA A. et al., 1995, Phytochemistry, 39, 771-774
(2) GORDON D. et al., 1992, J. Am. Chem. Soc., 114, 659-663
(3) GUSTAFSON K. R. et al., 1989, J. Nat. Cancer Inst. MD, vol. 81, 16, 1254-1258
(4) LOYA S. et al., 1998, J. Nat. Prod., 61, 891-895
(5) BLOCH E. G. et al., 1959, Can. J. Biochem. Physiol., 37, 911-917
(6) LEPAGE M., 1967, Lipids, 2, 244-250
(7) DEMANDRE C. et al., 1985, Phytochemistry, 24, 481-485
(8) DEMANDRE C. et al., 1986, Biochim. Biophys. Acta, 877, 380-386
(9) VIOQUE E., 1984, in “Handbook of chromatography lipids”, H. K. Mangold ed., vol. 11, 309-329, CRC Press Inc.
(10) METCALFE L. D. et al., 1966, Anal. Chem., 38, 514-515
(11) PHAM QUOC K. et al., 1994, Biochim. Biophys. Acta, 1168, 94-99
(12) PHAM QUOC K. et al., 1994, Plant Physiol. Biochem., 32, 501-509
(13) PHAM QUOC K. et al., 1997, Biochim. Biophys. Acta, 1346, 237-246
(14) FISHER W. E. et al., 1973, Hoppe-Seyler Z. Physiol. Chem., 354, 1115-1123
(15) NORMAN H. A. et al., 1996, J. Lipid Res., 37, 1372-1376
(16) CLARK P. K. et al., 1990, AIDS Res. Hum. Retroviruses, 753-764
(17) HIZI A. et al., 1988, Proc. Natl. Acad. Sci. USA, 85, 1218-1222
(18) HIZI A. et al., 1991, Virology, 180, 339-346
(19) LOYA S. et al., 1992, Arch. Biochem. Biophys., 293, 208-212
(20) HIZI A. et al., 1991, J. Biol. Chem., 1991, 266, 6230-6239
(21) LOYA S. et al., 1998, J. Nat. Prod., 1998, 61, 891-895

1. Spirulin biomass rich in sulfolipids, having a proportion of said sulfolipids of at least 40% by weight, based on total lipids, said sulfolipids having an inhibitory activity towards HIV-1 and HIV-2 reverse transcriptase.

2. Biomass according to claim 1, characterized in that said sulfolipids have formula (I) below: 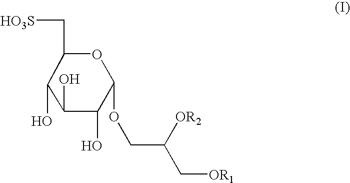
in which R₁ and R₂ are as defined as follows:
R₁ is an oleoyl radical and R₂ is a palmitoyl radical, or
R₁ is a linoleoyl radical and R₂ is a palmitoyl radical, or
R₁ is a palmitoyl radical and R₂ is a palmitoyl radical, or
R₁ is a γ-linolenoyl radical and R₂ is a palmitoyl radical, or
R₁ is a γ-linolenoyl radical and R₂ is a palmitoleoyl radical, or
R₁ is a palmitoleoyl radical and R₂ is a palmitoyl radical, or
R₁ is an oleoyl radical and R₂ is a linoleoyl radical, or
R₁ is a linoleoyl radical and R₂ is an oleoyl radical, or
R₁ is a linoleoyl radical and R₂ is a linoleoyl radical, or
R₁ is an oleoyl radical and R₂ is an oleoyl radical.

3. Biomass according to claim 1 or 2, characterized in that the spirulin is selected from the species consisting of Spirulina platensis PCC 8005, Spirulina maxima, Spirulina texcoco, Spirulina crater and Spirulina 8818.

4. Process for the mixotrophic culture of spirulins for the production of a biomass rich in sulfolipids according to claim 1, characterized in that said production comprises at least one step for the culture of spirulins in the presence of ammonium oleate or palmitate.

5. Prokaryotic sulfolipids of formula (I): 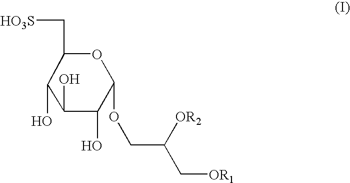
in which:
R₁ is a γ-linolenoyl radical and R₂ is a palmitoyl radical, or
R₁ is a γ-linolenoyl radical and R₂ is a palmitoleoyl radical,
or mixtures thereof.

6. Eukaryotic sulfolipids of formula 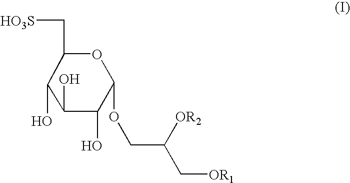
in which:
R₁ is a C18 unsaturated fatty acid radical and R₂ is a C18 unsaturated fatty acid radical, said radicals being identical or different, or mixtures thereof.

7. Eukaryotic sulfolipids of formula (I): (I) 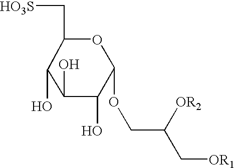
in which:
R₁ is an oleoyl radical and R₂ is a linoleoyl radical, or
R₁ is a linoleoyl radical and R₂ is an oleoyl radical, or
R₁ is a linoleoyl radical and R₂ is a linoleoyl radical, or
R₁ is an oleoyl radical and R₂ is an oleoyl radical, or mixtures thereof.

8. Total sulfolipids, consisting essentially of of a mixture of prokaryotic sulfolipids as defined in claim 5.

9. A reverse transcriptase inhibitor comprising a sulfolipid according to 5.

10. An HIV-1 or HIV-2 reverse transcriptase inhibitor comprising a spirulin biomass rich in sulfolipids according claim 1.

11. Pharmaceutical composition containing the sulfolipids according to claim 5 in association with a pharmaceutically acceptable vehicle.

12. A drug for prophylactic or curative treatment of AIDS comprising sulfolipids according to claim 5.

13. A drug for prophylactic or curative treatment of AIDS comprising Use of the spirulin biomass rich in sulfolipids according to claim 1.
